(19) United States
(12) Patent Application Publication
(10) Pub. No.: US 2008/0255090 A1
(43) Pub. Date: Oct. 16, 2008

(54) Ceratamines A and B, and Analogues, Syntheses and Pharmaceutical Compositions Thereof

(75) Inventors: Raymond J. Andersen, Vancouver (CA); Michel Roberge, Vancouver (CA); Emiliano Manzo, Pozzuoili (IT)

Correspondence Address: ARENT FOX LLP, 1050 CONNECTICUT AVENUE, N.W., SUITE 400, WASHINGTON, DC, 20036, US

(73) Assignee: THE UNIVERSITY OF BRITISH COLUMBIA, Vancouver, BC (CA)

(21) Appl. No.: 10/571,932

(22) PCT Filed: Sep. 15, 2004
(86) PCT No.: PCT/CA2004/001680

§ 371 Date: Jul. 3, 2008

Related U.S. Application Data
(60) Provisional application No. 60/502,627, filed on Sep. 15, 2003.

(51) Int. Cl. C07D 487/04; A61K 31/55; A61P 35/00

(52) U.S. Cl. 514/212.06; 540/523; 435/375

(57) ABSTRACT

Antimitotic compounds and salts are provided in which the compound has the structure (formula (I)): wherein X and Y are substituted or unsubstituted and are selected from carbon atoms and atoms of groups 15 and 16 of the periodic table; Z is selected from N—R, O and S; U is selected from CR¹, N, NH, NR, S and O, R¹, R², and R³ are independently selected from H, NH₂, NHR, NR₂, SH, SR, SiR₃, OH, OR, F, CI, Br, I, ═O, ═S and R; W is selected from O, S, and H₂; R⁴ is selected from H, R, OH, OR, SH, SR, NH₂, NHR, NR₂ and halide; R is selected from H, substituted or unsubstituted aryl, and substituted or unsubstituted 1 to 20 carbon linear, branched, or cyclic, saturated or unsaturated alkyl, in which alkyl carbon atoms are replaced by 0 to 10 oxygen, 0 to 10 sulphur, and 0 to 10 N atoms; and wherein bonds to carbon atoms or to any of X, Y, Z and U are saturated or unsaturated; and providing that when W is H₂, R⁴ is not H, OH, or unsubstituted phenyl.

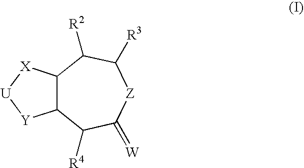

### BACKGROUND

Antimitotic drugs based on natural products in the taxane (paclitaxel and docetaxel) and Vinca alkaloid (vincristine, vinblastine, vinorelbine) families are widely used in the treatment of cancer. The effectiveness of these drugs has stimulated the search for other natural products that arrest cancer cell-cycle progression in mitosis. Marine invertebrates have been a particularly rich source of antimitotic secondary metabolites. Included in this group are discodermolide, dolastatin-10, laulimalide, eleutherobin, halichondrin B, peloruside, vitilevuamide, spongistatin, and hemiasterlin.

### SUMMARY OF THE INVENTION

This invention provides novel compounds suitable for use as antimitotic agents and as intermediates for the production of antimitotic agents.

Various embodiments of this invention provide a substantially purified or isolated compound or salt thereof, having the structure of formula I

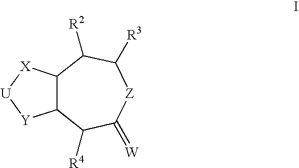

wherein X and Y are substituted or unsubstituted and are selected from carbon atoms and atoms of groups 15 and 16 of the periodic table (also known as group 5B or VA and group 6B or VIA, respectively);
Z is selected from N—R, O and S;
U is selected from CR¹, N, NH, NR, S and O;
R¹, R², and R³ are independently selected from H, NH₂, NHR, NR₂, SH, SR, SiR₃, OH, OR, F, Cl, Br, I, ═O, ═S and R;
W is selected from O, S, and H₂;
R⁴ is selected from H, R, OH, OR, SH, SR, NH₂, NHR, NR₂ and halide;
R is selected from H; substituted or unsubstituted aryl; and substituted or unsubstituted 1 to 20 carbon linear, branched, or cyclic, saturated or unsaturated alkyl, wherein alkyl carbon atoms are replaced by 0 to 10 oxygen, 0 to 10 sulphur, and 0 to 10 N atoms;
and wherein bonds to carbon atoms or to any of X, Y, Z and U in formula I are saturated or unsaturated; and providing that when W is H₂, R⁴ is not H, OH, or unsubstituted phenyl.

Various other embodiments of this invention also provides the use of an antimitotic compound of this invention or a pharmaceutical or physiologically acceptable salt thereof, as an antimitotic agent and for the preparation of antimitotic agents and compositions, including medicaments. This invention also provides a method for causing mitotic arrest in one or more cells present in a cell population, comprising treating the cell population with a sufficient amount of an antimitotic compound, salt, or composition of this invention to arrest mitosis in one or more cells in the cell population. The cell population may comprise proliferative cells, including tumor cells. This method may be performed in vitro (such as in cytological procedures) or may be performed in vivo in a human or animal patient.

### DETAILED DESCRIPTION OF INVENTION

As part of a program to discover new antimitotic natural products, extracts of marine invertebrates are screened in a cell-based assay that detects mitotic arrest (Roberge, M. et al. (2000) Cancer Res. 60:5052). Extracts of the marine sponge Pseudoceratina sp. collected in Papua New Guinea showed activity in the assay. Bioassay guided fractionation of the extract resulted in the isolation of ceratamines A and B, two novel antimitotic heterocyclic alkaloids, whose structures are reported below. The compounds described below, their salts, prodrugs and pharmaceutical compositions thereof are all contemplated within the scope of this invention.

In its broadest embodiment the invention contemplates compounds and salts that contain the following core heterocycle:

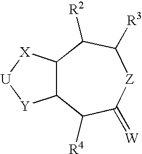

wherein the atoms X and Y are substituted or unsubstituted C or atoms independently selected from groups 15 and 16 of the periodic table and are preferably CR¹, N, NH, NR, O, S, or P and are most preferably nitrogen atoms. Z is N—R, O or S, and in preferred embodiments Z=N—R. U is CR¹, N, NH, NR, S, or O, and, in preferred embodiments U═N, NH, NR, or CR¹. Most preferably, U is CR¹, such as —CNRH and —CNH₂.
R¹ is selected from the group consisting of H, NH₂, NHR, NR₂, SH, SR, SiR₃, OH, OR, F, Cl, Br, I, ═O, ═S, or R.
R² and R³ are independently selected from the group: H, NH₂, NHR, NR₂, SH, SR, SiR₃, OH, OR, F, Cl, Br, I, ═O, ═S, or R.
W is O, S, or H₂, more preferably O or S, and most preferably O.
R⁴ is selected from the group: H, R, OH, OR, SH, SR, NH₂, NHR, NR₂, or halide. Preferably, it is a branched or otherwise bulky alkyl or an aryl group.
R may be hydrogen, an optionally substituted aryl group, or an optionally substituted 1 to 20 carbon linear or branched or cyclic, saturated or unsaturated alkyl group where the carbon atoms of the alkyl backbone are optionally and independently substituted with (CH₂)ₚH, O(CH₂)ₚH or OC(O)(CH₂)ₚH groups (p are independently=0 to 10), interrupted by 0 to 10 oxygen (O) atoms, 0 to 10 sulphur (S) atoms (e.g. as S, SO, SO₂) or by secondary amino (NR⁵) groups, and/or substituted by a zero to (2p+1) number of aryl, hydroxyl, thio, thioether, amino, halide, ester, amide, carbonyl, thiocarbonyl, carboxyl and/or carboxylate groups. R⁵ is hydrogen, an optionally substituted linear or branched alkyl group or optionally substituted aryl group, where optional substitution of R⁵ refers to the presence of substituents selected from ether, amino, hydroxy, ester, thioether, amide, carbonyl, thiocarbonyl, carboxyl, carboxylate, sulphate, nitro, and halide groups.

Where R is an aryl group, or is substituted by an aryl group, the aryl group can be optionally substituted with alkyl, aryl, acyl, ether, amino, hydroxy, ester, thioether, amide, nitro, carbonyl, carboxyl, carboxylate, and halide groups.

Where a substituent in the definition of R above is amino, it may have the definition of R⁵ above.

The backbone shown in the general structure above may be fully saturated. However, unsaturation of the any of the carbon-carbon single bonds or C—X, C—Y, C-Z, UX, or UY single bonds to form double bonds ((C═C, C═X, C═Y, C=Z, U═X, U═Y) is contemplated within the scope of this invention. In fact, when X, Y are nitrogen, Z is N—R, and W is not H₂, a preferred embodiment is the formally aromatic general structure shown below:

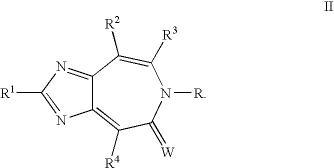

Hydrogenated derivatives, in which one or more of the sites of unsaturation in the structure shown above may be independently reduced to their saturated analogues, are also contemplated within the scope of this invention.

More specific preferred embodiments of the contemplated invention include the following general structures

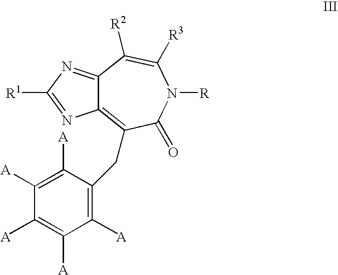

wherein A groups are independently selected from H and alkyl, aryl, acyl, ether, halide, thioether, hydroxyl, amino, nitro, cyano, and carboxylate substituents. In preferred embodiments R¹ in the above structure is an amine group. In specific preferred embodiments the para-A substituent is —OR or —OH, most preferably —OMe, and the meta-A substituents are halides, most preferably bromide.

Specific preferred embodiments of the invention utilize either ceratamine A (1) or ceratamine B (2) shown below, in which Me is methyl.

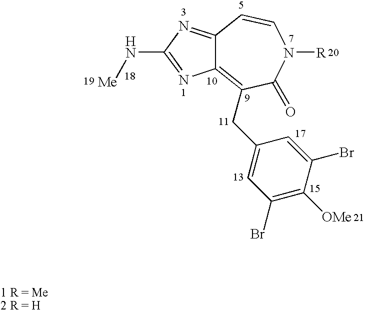

Compounds of this invention are contemplated as being useful when isolated or substantially purified. The term substantially purified means either that the compound has been synthesized or if obtained from natural sources, the compound is in a form that is at least two-fold purified over that present in the most crude cellular extract. Preferably naturally occurring compounds will be purified at least five fold over crude extracts.

Some ceratamines can be isolated from natural sources. As well, compounds of this invention can be synthesized via the following general biomimetic and chemical routes, also illustrated below for desmethylceratamine B, ceratamine B, and ceratamine A. Naturally occurring compounds can also be modified by routine procedures to produce analogues within the scope of this invention. It will be obvious to one skilled in the art that the synthetic procedures can be readily adapted to the synthesis of a number of ceratamine analogues.

The following biomimetic routes follow what may be the actual biogenesis pathway for ceratamines A and B and is based on methodologies known in the art (for example, as disclosed in Bates, G. S. and Ramaswany (1980) Can. J. Chem. 58:716; Marchais, S. et al. (1998) Tetrahedron Lett. 39:8085; Wang, X. and Porco, J. A. (2001) J. Org. Chem. 66:8215; and Nagai, W. et al. (1973) 11:1971). The following chemical synthesis scheme is based on known methodologies as disclosed in Nicolaou, K. C. et al. (2002) J. Am. Chem. Soc. 124:5718; Humphries, Mark E. et al. (2003) J. Org. Chem. 68:2432; and Rajendra, G. and Miller, M. J. (1987) J. Org. Chem. 52:4471).

In these schemes, acronyms have the following meanings: OTs is tosylate; DCC is 1,3,dicyclohexylcarbodiimide; DIEA is N,N′-diiospropylethylamine; THF is tetrahydrofuran; Me is methyl; and MeI is iodomethane.

### General Biomimetic Synthesis Route #1

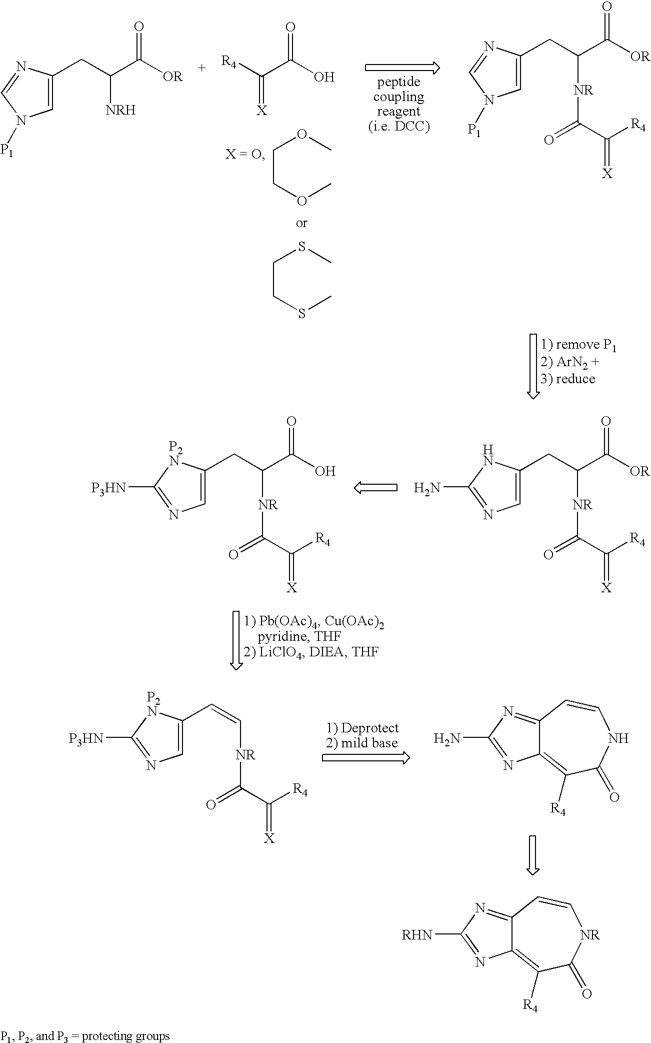

### Example of Biomimetic Synthesis Route #1

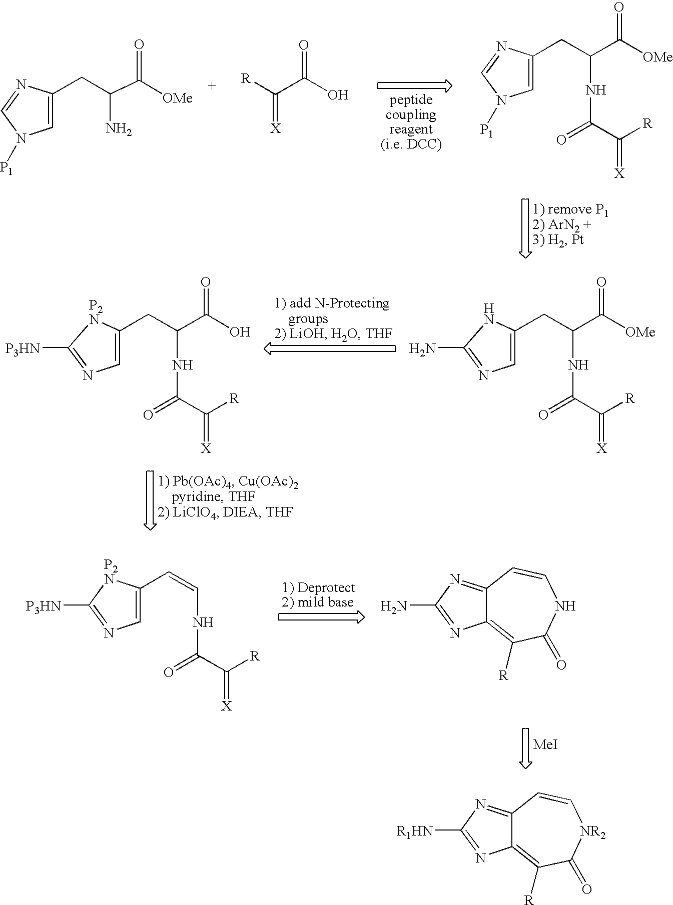

### General Biomimetic Synthesis Route #2

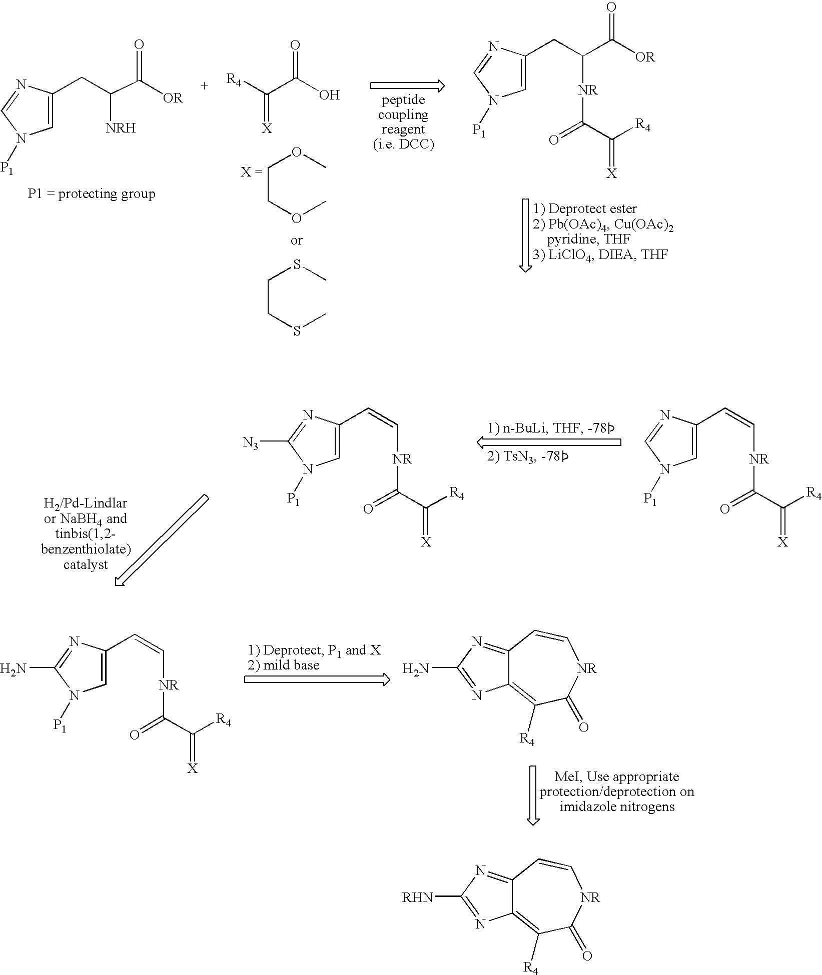

### Example of Biomimetic Route #2

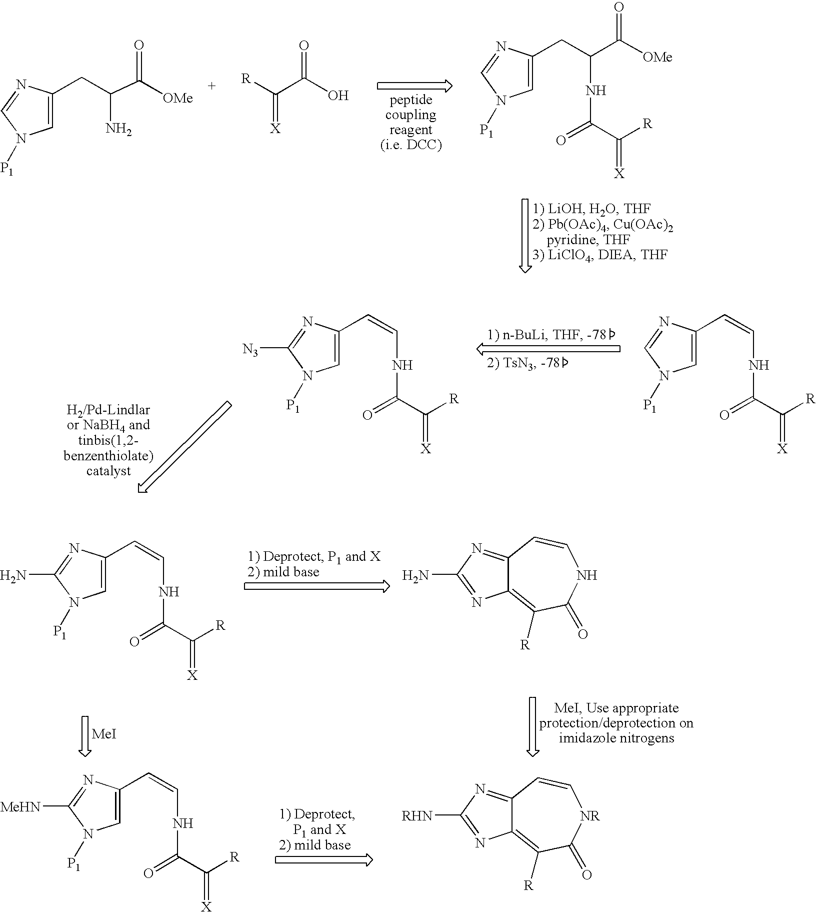

### Example of Synthesis of Desmethylceratamine B via Biomimetic Route #1

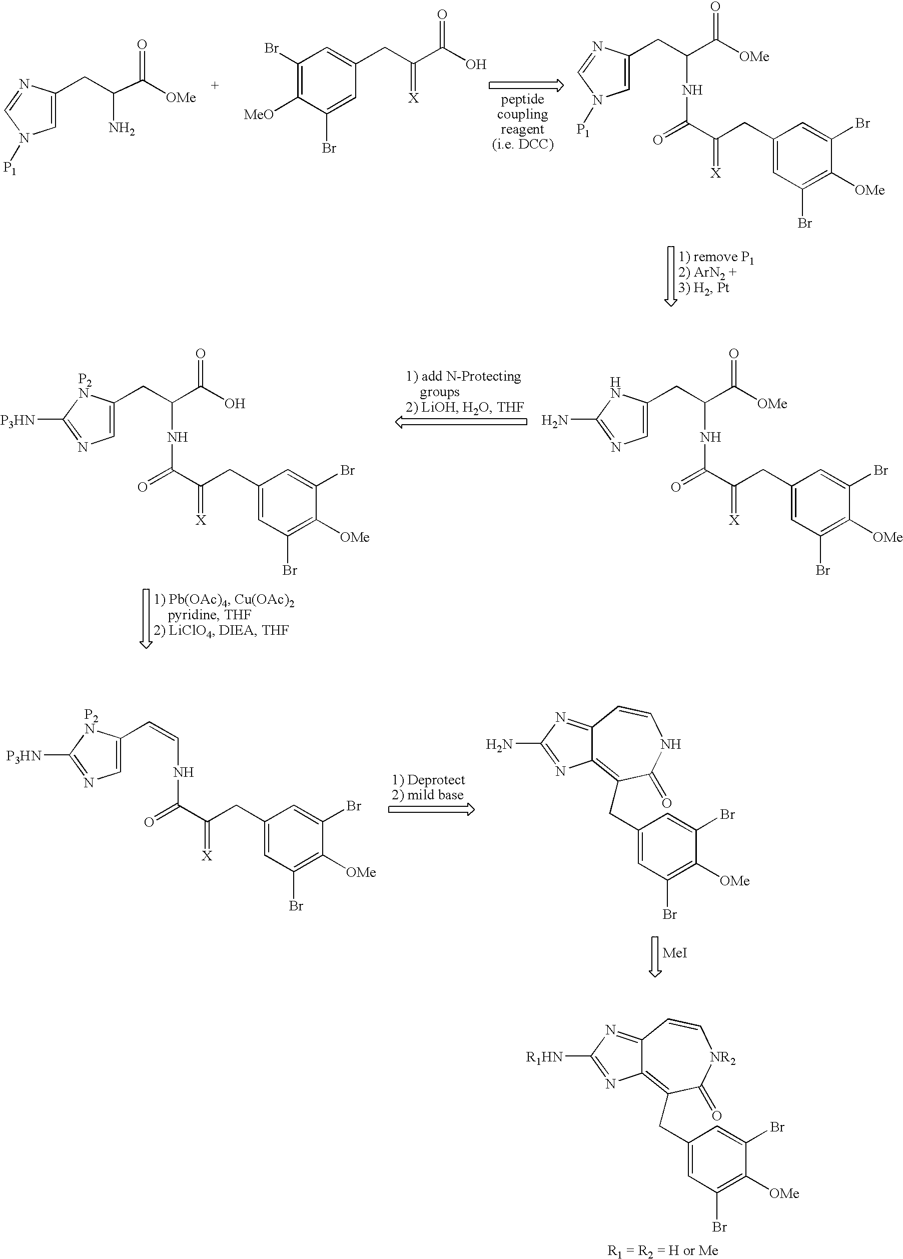

### Example of Synthesis of Ceratamine B and Ceratamine A via Biomimetic Route #2

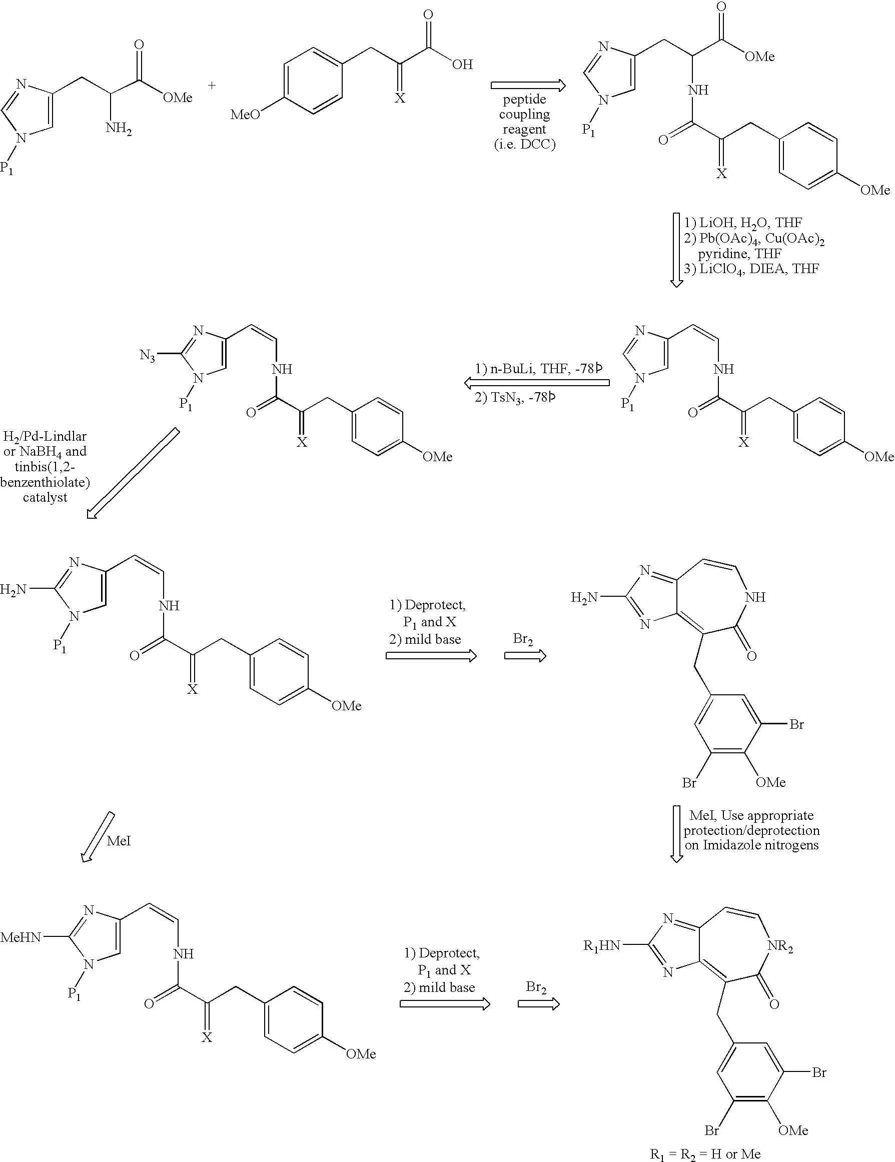

### General Chemical Synthesis to Make Broad Spectrum of Analogs

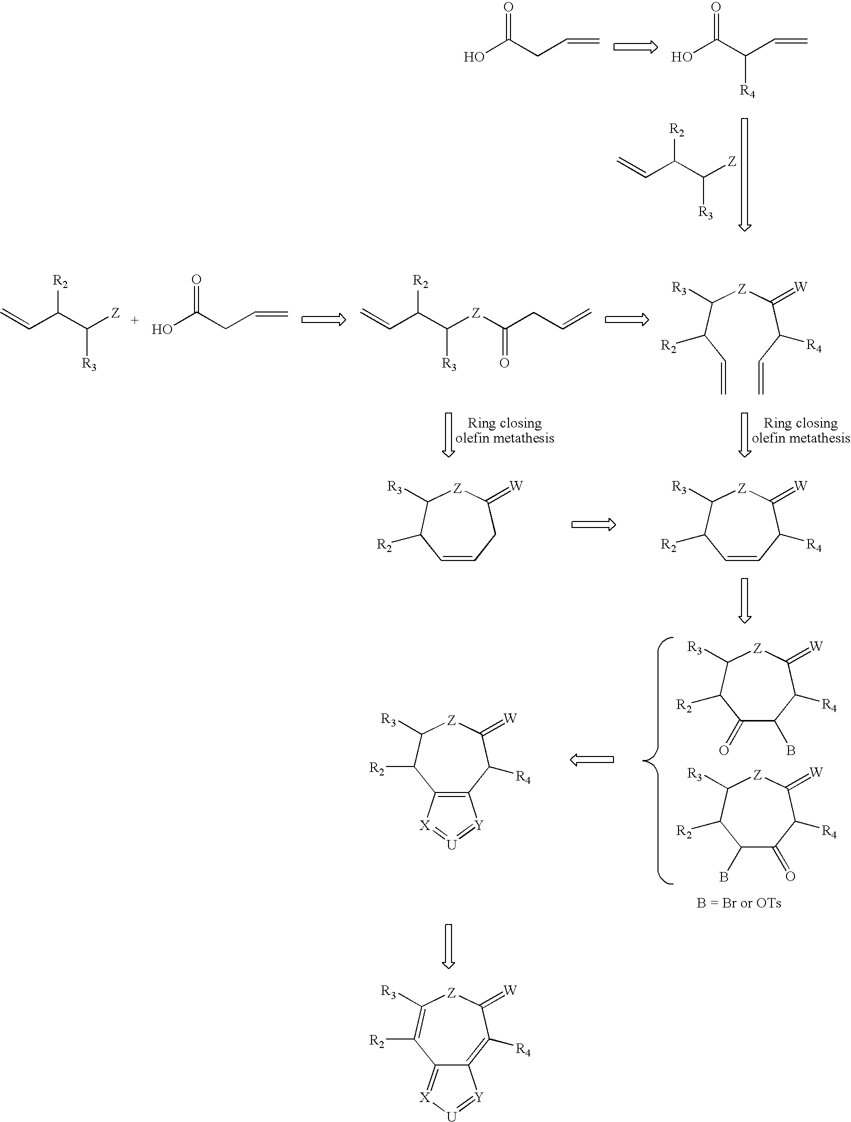

### Example of Chemical Synthesis Applied to Ceratamine A

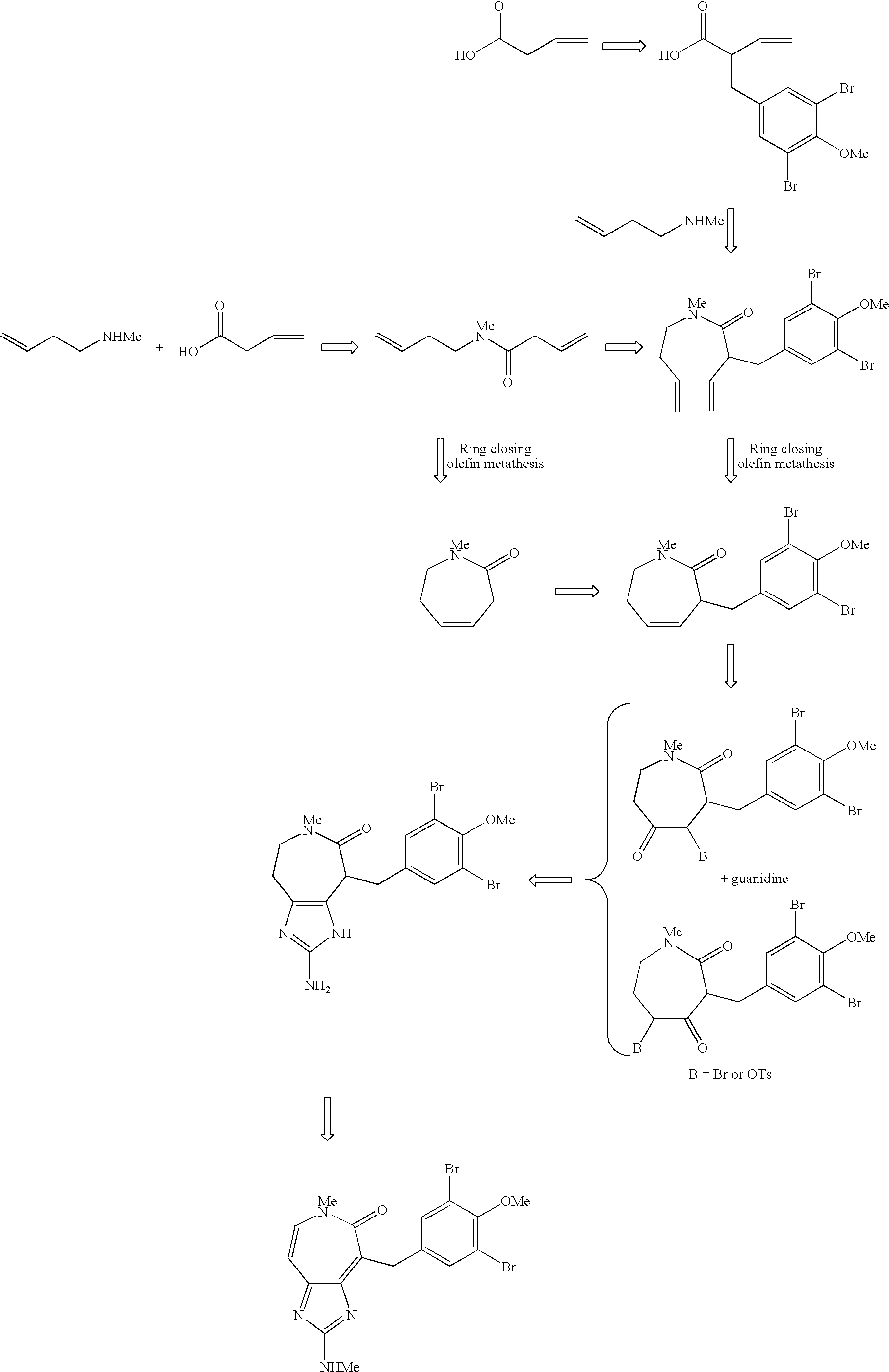

Compounds of this invention include those having improved solubility because of the presence of polar substituents (for example, carboxyl or hydroxyl groups on R⁴) and/or being present in a salt form such as acid addition salts, which make use of ammonium counterions in the compound derived from amine groups. Such polar groups may include ionizable groups, which would also facilitate the formation of salts, such as pharmaceutically acceptable acid or base addition salts. Increasing the solubility of antimitotic compounds of this invention facilitates formulation of the compounds thereby permitting one to avoid difficulties associated with formulation of hydrophobic drugs.

Compounds of this invention may also be modified to increase the reactivity of the compound, thereby permitting compounds of this invention to function as intermediates in the preparation of compounds in which other chemical moieties are joined to the compound. Examples of moieties which increase the reactivity of the compound for joining to other moieties while retaining stability include: —CNH₂ and —COOH. The latter substituents are ideally suited for joining to amine or carboxylic acid-containing moieties by means of a peptide linkage.

Compounds of this invention may have a substituent which comprises a linker which in turn may be used for conjunction to another functional group. Such a linker may be any linker known in the art for joining biologically active compounds or for joining a biologically active compound to a carrier. Such linkers may be cleavable upon the action of an agent present at or near a target site (e.g. reduced pH) or which is administered in conjunction with the compound of this invention. An example of such linkers are those described by Czerwinski, et al. (1998) Proc. Natl. Acad. Sci. 95:11520-11525, in WO 89/11867 and WO 91/12023, or the metal chelating linkages described in WO 00/64471, WO 01/28569, and U.S. Pat. No. 6,087,452.

Compounds of this invention include compounds intended for use as intermediates, for example, by joining to another chemical moiety. Thus, moieties to be added to compounds of this invention may be any functional group or moiety selected to provide a desired performance in vivo. Without limitation, these may comprise a peptide (including polypeptides and proteins), a lipid, a polysaccharide, a pharmaceutically compatible polymer or another drug. Thus, this invention include compounds that are conjugated to a lipid in a lipid-based delivery vehicle such as a liposome, to a peptide that facilitates transfer across a cell membrane, to an antibody having specificity for a target cell and to a peptide ligand capable of binding to a cell surface receptor or the like. In other embodiments, a polymer which facilitates pharmaceutical preparation or which enhances delivery of an active compound within the body may be used.

The physical, chemical, or biological characteristics of a compound of this invention can be altered in many ways that would be apparent to persons skilled in the art. Different functional groups will alter solubility through the addition of groups that for example alter polarity and/or the ability to form hydrogen bonds. Similarly a functional group may alter the stability by changing the serum half-life or by controlling the release of the compound from a micelle at the target site or converting a prodrug to the active form at the target site. Further a functional group may alter biocompatibility, for example by minimizing the side effects of the drug to the patient. A functional group may further enhance delivery and targeting through the addition of a functional group capable of binding the target cells or tissues or facilitating the transport into the target cells. The functional group may also enhance the anti-tumor activity of the compound if for example the compound is conjugated to another anti-proliferative drug. A person skilled in the art will appreciate what type of functional groups might be added to achieve the desired result in administering the compound to the patient and thereby improving the overall therapeutic index.

A functional group conjugated to a compound of this invention may be a biological targeting molecule that binds to a specific biological substance or site. The biological substance or site is the intended target of the delivery and targeting molecule that binds to it, enabling the delivery of the compound to the tissue or cells of interest.

A ligand may function as a biological targeting molecule by selectively binding or having a specific affinity for another substance. A ligand is recognized and bound by a specific binding body or binding partner, or receptor. Examples of ligands suitable for targeting are antigens, haptens, biotin, biotin derivatives, lectins, galactosamine and fucosylamine moieties, receptors, substrates, coenzymes and cofactors among others. A ligand may include cancer and tumor antigens such as alpha-fetoproteins, prostate specific antigen (PSA) and CEA, cancer markers and oncoproteins, among others. Other substances that can function as ligands for delivery and targeting are certain steroids, prostaglandins, carbohydrates, lipids, certain proteins or protein fragments (i.e. hormones, toxins), and synthetic or natural polypeptides with cell affinity. Ligands also include various substances with selective affinity for ligators that are produced through recombinant DNA, genetic and molecular engineering.

Another type of targeting molecule is an antibody, which term is used herein to include all classes of antibodies, monoclonal antibodies, chimeric antibodies, Fab fractions, fragments and derivatives thereof. Other targeting molecules include enzymes, especially cell surface enzymes such as neuraminidases, plasma proteins, avidins, streptavidins, chalones, cavitands, thyroglobulin, intrinsic factor, globulins, chelators, surfactants, organometallic substances, staphylococcal protein A, protein G, cytochromes, lectins, certain resins, and organic polymers. Targeting molecules may include peptides, including proteins, protein fragments or polypeptides which may be produced synthetically or through recombinant techniques known in the art. Examples of peptides include membrane transfer proteins which could facilitate the transfer of the compound to a target cell interior or for nuclear translocation (see: WO 01/15511).

Other examples of moieties which may facilitate transfer into a target cell are described in U.S. Pat. No. 6,204,054, which includes transcytosis vehicles and enhancers capable of transporting physiologically-active agents across epithelia, endothelia and mesothelia containing the GP60 receptor. The GP60 receptor has been implicated in receptor-mediated transcytosis of albumin across cell barriers. U.S. Pat. No. 6,204,054 exploits GP60 receptor-mediated transcytosis for the transport of physiologically-active agents which do not naturally pass through epithelia, endothelia and mesothelia via the GP60 system. The compound can be coupled to albumin, albumin fragments, anti-GP60 polyclonal and monoclonal antibodies, anti-GP60 polyclonal and monoclonal antibody fragments, and GP60 peptide fragments to facilitate transport into the cell.

Conjugation to a functional group may also improve other properties of a compound of this invention. Such functional groups may be termed drug carriers and can improve the solubility, stability, or biocompatibility of the drug. For example the solubility may be improved by conjugation to a peptide polymer. By way of example U.S. Pat. Publication No. 2001041189 describes the use of polypeptides (containing glutamic acid and aspartic acid, or glutamic acid/alanine, or glutamic acid/asparagine, or glutamic acid/glutamine, or glutamic acid/glycine) conjugated to hydrophobic drugs such as paclitaxel to act as carriers to improve the solubility of the drugs and/or their therapeutic efficacy in vivo. Similarly, U.S. Pat. No. 5,087,616 describes the use of a biodegradable polymeric carrier (a homopolymer of polyglutamic acid) to which one or more cytotoxic molecules, such as daunomycin is conjugated. Also by way of example, U.S. Pat. No. 4,960,790 describes paclitaxel covalently conjugated to an amino acid (glutamic acid) to improve drug solubility. Another example is described in U.S. Pat. No. 5,420,105, where polypeptide carriers capable of binding one drug or multiple drugs can further be attached to a targeting or delivery protein, such as an antibody or ligand capable of binding to a desired target site in vivo.

Another example of a drug carrier is described in U.S. Pat. Publication No. 2001034333, where cyclodextrin polymers are used for carrying drugs and other active agents for therapeutic, medical or other uses. The 2001034333 specification also discloses methods for preparing compositions of cyclodextrin polymer carriers that are further coupled to delivery and targeting molecules to deliver drugs, like paclitaxel and doxorubicin, to their site of action.

By way of a further example, U.S. Pat. No. 6,127,349 describes the use of phospholipids to improve the solubility of therapeutic agents(steroids, peptides, antibiotics and other biologically active agents and pharmaceutical formulations) and to improve their bio-availability. Similarly, fatty acids could be conjugated to the compound in order to stabilize the activity of the anti-angiogenic substances. By way of example U.S. Pat. No. 6,380,253 describes the conjugation of anti-angiogenic substances (proteins—angiostatin and endostatin etc.) to cis-unsaturated fatty acids or polyunsaturated fatty acids to potentiate and stabilize the activity of the anti-angiogenic substances.

Other suitable drug carriers include biologically compatible polymers such as polyethylene glycol (PEG) and related polymer derivatives. Drug-PEG conjugates have been described as improving the circulation time (prolong serum half-life) before hydrolytic breakdown of the conjugate and subsequent release of the bound molecule thus increasing the drugs efficacy. For example, U.S. Pat. No. 6,214,966 describes the use of PEG and related polymer derivatives to conjugate to drugs such as proteins, enzymes and small molecules to improve the solubility and to facilitate controlled release of the drug. Alternatively, EP 1082105 (WO 99/59548) describes the use of biodegradable polyester polymers as a drug delivery system to facilitate controlled release of the conjugated drug.

As another alternative a compound of this invention may be conjugated to another pharmaceutically active compound to enhance the therapeutic effect on the target cell or tissue by delivering a second compound with a similar anti-mitotic effect or a different activity altogether. For example, U.S. Pat. No. 6,051,576 describes the use of co-drug formulations by conjugating two or more agents via a labile linkage to improve the pharmaceutical and pharmacological properties of pharmacologically active compounds.

Methods suitable for assaying antimitotic activity of compounds of this invention or compounds used as precursors, may be based on any of a variety of known methodologies including microscopic examination of cells to determine if they are arrested in mitosis. Particularly useful methods employ antibodies specific for mitotic cells, such as the method described in the international patent application published under WO 99/15157 or the assays described in WO 01/38339 and in Roberge et al. (2000) Cancer Res. 60:5052. Assays will typically employ cells which regularly divide in culture (e.g. cancer cells). A known antimitotic compound such as nocodazole may be used as a control. In immunological procedures, determination of the cells which proceed to mitosis may be carried out using any of the known immunological methods by employing antibodies which have specificity for mitotic cells. Monoclonal antibodies demonstrating such specificity are known and include MPM-2 which was raised against mitotic HeLa cells and recognizes phospho-epitopes that are highly conserved in mitotic proteins of all eukaryotic species. Other examples are the monoclonal antibodies recognizing phospho-epitopes in the paired helical filament proteins (PHF) found in brain tissue of patients suffering from Alzheimer's disease as described in: PCT International Application published Jul. 4, 1996 under No. WO 96/20218; and, Vincent et al. (1996) “The Journal of Cell Biology”, 132:413-425. TG-3 antibody described in the latter two references may be obtained from Albert Einstein College of Medicine of Yeshiva University, Bronx, N.Y. This antibody is highly specific for mitotic cells and functions in ELISA.

Immunological methods useful for determination of mitotic cells in an assay include any method for determining antibody-antigen binding, including: immunocytochemistry (e.g. immunofluorescence), flow cytometry, immunoblotting, and ELISA, including those described in Vincent, I. et al. [supra]. High throughput testing of samples may be readily achieved by use of the ELISA or the ELICA assays described in WO 01/38339.

Pharmaceutical preparations containing compounds of this invention may be prepared as for similar preparations containing eleutherobin, paclitaxel, etc. In the case of compounds of this invention capable of salt formulation, pharmaceutically or physiologically acceptable salts may be used to advantage to permit administration of the compound in an aqueous solvent. Modes of administration to an animal or human patient include intravenous and intraperitoneal, to achieve a circulating concentration of the drug as predicted from its activity using standard methodology.

A human or other animal patient suffering from proliferative diseases, and other similar conditions may be treated by administering to the patient an effective amount of one or more of the compounds of this invention or a pharmaceutically acceptable derivative or salt thereof, in a pharmaceutically acceptable carrier or diluent. The active materials can be administered by any appropriate route, for example, orally, parenterally, intravenously, intradermally, or subcutaneously.

The term pharmaceutically or physiologically acceptable salts or derivatives refers to salts or complexes that retain the antimitotic activity of the compound and exhibit minimal undesired toxicological effects. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid; (b) base addition salts formed with polyvalent metal cations such as zinc, calcium, bismuth, barium, magnesium, aluminum, copper, cobalt, nickel, cadmium, sodium, potassium, and the like, or with an organic cation formed from N,N-dibenzylethylene-diamine, D-glucosamine, ammonium, tetraethylammonium, or ethylenediamine; or (c) combinations of (a) and (b); e.g., a zinc tannate salt or the like.

A compound of this invention or salt thereof, may be included in a pharmaceutically acceptable carrier or diluent, ideally in an amount sufficient to deliver to a patient a therapeutically effective amount without causing serious toxic effects in the patient treated. The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgement of the person administering or supervising the administration of the compositions.

Solutions or suspensions used for parenteral, intradermal, subcutaneous, or topical application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of toxicity such as sodium chloride or dextrose.

Suitable pharmaceutically acceptable carriers for parenteral application, such as intravenous, subcutaneous, or intramuscular injection, include sterile water, physiological saline, bacteriostatic saline (saline containing 0.9 mg/ml benzyl alcohol) and phosphate-buffered saline. If administered intravenously, preferred carriers are physiological saline or phosphate buffered saline.

Methods for preparing transdermal formulations including topical formulations or transdermal delivery devices such as patches are known to those skilled in the art. For example, see Brown L., and Langer R., Transdermal Delivery of Drugs (1988), Annual Review of Medicine, 39:221-229.

Compounds of this invention may be prepared with carriers that will protect the compound against rapid elimination from the body, such as through controlled release formulations, including implants and microencapsulated delivery systems. Biodegradable, biocompatible polymers can be used, such as ethylene vinyl acetate, polyanhydrides, polyglycolic acid, collagen, polyorthoesters, and polylactic acid.

Liposomal suspensions are also suitable carriers for compounds of this invention. The compounds may be conjugated to a lipid by known methods for incorporation into a liposomal envelope or the compounds may be encapsulated into the liposome. Liposomes may be prepared according to methods known to those skilled in the art, such as is described in U.S. Pat. No. 4,522,811. For example, liposome formulations may be prepared by dissolving appropriate lipid(s) (such as stearoyl phosphatidyl ethanolamine stearoyl phosphatidyl choline, arachadoyl phosphatidy choline, and cholesterol) in an inorganic solvent that is then evaporated, leaving behind a thin film of dried lipid on the surface of the container. An aqueous solution of the active compound or its monophosphate, and/or triphosphate derivatives are then introduced into the container. The container is then swirled by hand to free the lipid aggregates, thereby forming the liposomal suspension.

Oral compositions may include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. Methods for encapsulating compositions (such as in a coating of hard gelatin) for oral administration are well known in the art (Baker, Richard, Controlled Release of Biological Active Agents, John Wiley and Sons, 1986).

Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, or corn starch; a lubricant such as magnesium stearate; a glidant such as colloidal silicon dioxide; a sweetening agent such as sucrose or saccharin; or a flavouring agent such as peppermint, methyl salicylate, or orange flavouring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents. Alternatively, compounds of this invention could be administered as a component of an elixir, suspension, syrup, wafer, chewing gum or the like. A syrup may contain, in addition to the active compounds, sucrose as a sweetening agent and certain preservatives, dyes and colourings and flavours.

### EXAMPLES

Specimens of Pseudoceratina sp. were collected by hand using SCUBA at −15 m on the outer reefs near Motupore Island, Papua New Guinea. Freshly collected sponge (190 g) was frozen on site and transferred to Vancouver over dry ice. A voucher specimen (ZMAPOR 17512) has been deposited at the University of Amsterdam. Thawed sponge was extracted at room temperature with MeOH (3×0.5 L) and the combined MeOH extracts were evaporated in vacuo to give a gummy residue that was active in the antimitotic assay described below. The residue was partitioned between MeOH/H₂O (90:10) and hexanes, and then water was added to the MeOH phase (to reach 60:40 MeOH/H₂O) before extracting it with CH₂Cl₂. Fractionation of the antimitotic CH₂Cl₂ soluble materials via reversed phase flash chromatography (Sep-pak™ 10 g) eluting with a step gradient from H₂O to MeOH gave activity in the MeOH/H₂O (8:2) wash. This material was further purified via reversed phase HPLC eluting with MeOH/H₂O (8:2) to give ceratamines A (1) (8 mg) and B (2) (14 mg).

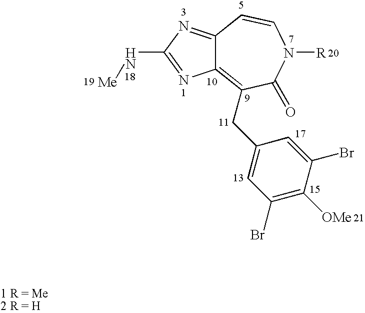

Ceratamine A (1) may be isolated as small yellow crystals from MeOH (mp 236° C.). The EIHRMS spectrum of 1 shows a molecular ion at m/z 467.9624 that corresponds to a molecular formula of C₁₇H₁₆⁷⁹Br⁸¹BrN₄O₂ (calcd 467.9620) requiring 11 sites of unsaturation. Although the number of resonances in the ¹H NMR spectrum of 1 is quite small (Table 1), the spectrum shows additional complexity due to the presence of signals for two slowly interconverting forms.

TABLE 1
NMR Data for Ceratamines A (1) and B (2) Recorded in
DMSO-d₆ at 500 MHz for ¹H and 100 MHz for ¹³C^{a}

| | A (1) | A (1) | B (2) | B (2) |
|---|---|---|---|---|
| C/N no. | δ ¹H | δ ¹³C | δ ¹H | δ ¹³C |
| 2 | | 175.6 | | 175.3 |
| 4 | | 160.5 | | 161.2 |
| 5 | | 121.3 | | 121.3 |
| 6 | | 164.1 | | 164.7 |
| 7 | | | 10.55, br s | |
| 8 | 7.73, d (10.0) | 142.9 | 7.14, t (9.5) | 137.9 |
| 9 | 6.42, d (10.0) | 100.4 | 6.42, d (9.5) | 101.2 |
| 10 | | 169.7 | | 170.8 |
| 11 | 4.23, s | 35.0 | 4.17, s | 33.9 |
| 12 | | 140.2 | | 140.1 |
| 13 | 7.67, s | 133.2 | 7.66, s | 133.1 |
| 14 | | 116.7 | | 116.7 |
| 15 | | 151.4 | | 151.4 |
| 16 | | 116.7 | | 116.7 |
| 17 | 7.67, s | 133.2 | 7.66, s | 133.1 |
| 18 | 8.69 | — | 8.69 | |
| 19 | 3.07, d (3.7) | 29.2 | 3.07, d (3.7) | 28.9 |
| 20 | 3.56, s | 43.7 | | |
| 21 | 3.71, s | 60.3 | 3.72, s | 60.3 |

^{a}The ¹³C assignments are based on HMQC and HMBC data. Only the shifts for the major tautomers are listed.

Tautomers of ceratamine A (1) are shown in Table 2. Each of the constitutional isomers II, III, and IV can exist as the E and Z stereoisomers about the C-2/N-8 imine bond.

TABLE 2

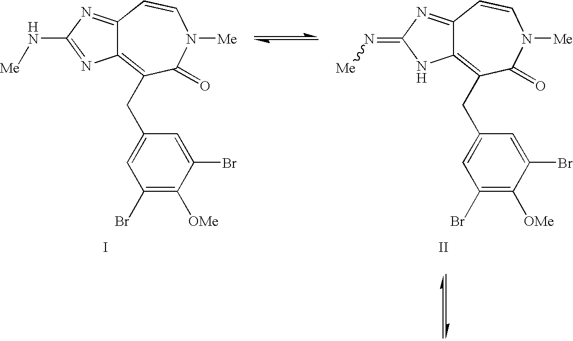

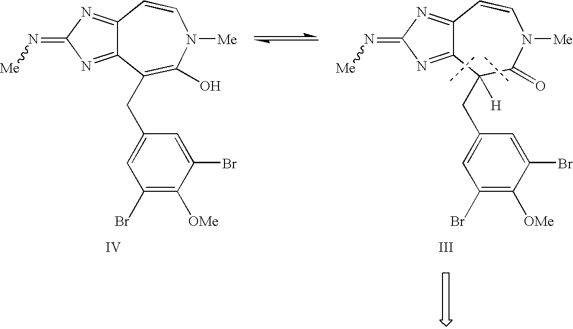

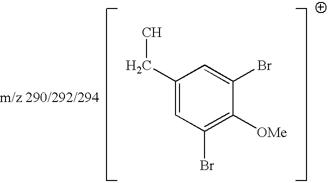

Ceratamine B (2) may be isolated as small yellow crystals from MeOH (mp 242° C.) that give a molecular ion in the HREIMS at m/z 453.9460 consistent with a molecular formula of C₁₆H₁₄⁷⁹Br⁸¹BrN₄O₂ (calcd 453.9463) that differs from the molecular formula of 1 only by the loss of CH₂. The 1D and 2D ¹H and ¹³C NMR data obtained for 2 show a strong resemblance to the data for 1, including evidence for the presence of multiple interconverting forms (Table 1). Routine analysis of the NMR data for 2 shows that it differs from 1 simply by replacement of the methyl substituent (ME-20) on N-7 with a proton.

Ceratamines A (1) and B (2) represent the first members of a new family of antimitotic sponge alkaloids. They have an unprecedented imidazo[4,5,d]azepine heterocyclic core structure. Heterocycles 1 and 2 exhibit potent activity (IC₅₀ 10 μg/ml) in the cell-based ELICA assay of Roberge et al. (2000) Cancer Res. 60:5052.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims. All patents, patent applications and publications referred to herein are hereby incorporated by reference.

1. A substantially purified or isolated compound or salt thereof, having the structure of formula 1 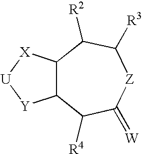
wherein X and Y are substituted or unsubstituted and are selected from carbon atoms and atoms of groups 15 and 16 of the periodic table;
Z is selected from N—R, O and S;
U is selected from CR¹, N, NH, NR, S and O;
R¹, R², and R³ are independently selected from H, NH₂, NHR, NR₂, SH, SR, SiR₃, OH, OR, F, Cl, Br, I, ═O, ═S and R;
W is selected from O, S, and H₂;
R⁴ is selected from H, R, OH, OR, SH, SR, NH₂, NHR, NR₂ and halide;
R is selected from: H; substituted or unsubstituted aryl; and, substituted or unsubstituted 1 to 20 carbon linear, branched, or cyclic, saturated or unsaturated alkyl in which alkyl carbon atoms are replaced by 0 to 10 oxygen, 0 to 10 sulphur, 25 and 0 to 10 N atoms;
wherein bonds to carbon atoms or to any of X, Y, Z and U in formula I are saturated or unsaturated; and
with the proviso that when W is H₂, R⁴ is not H, OH, or unsubstituted phenyl.

2. The compound or salt of claim 1, wherein W is O or S.

3. The compound or salt of claim 1, wherein W is O.

4. The compound or salt of claim 1, wherein U is N, NH, or NR.

5. The compound or salt of claim 1, wherein U Is —CR¹.

6. The compound or salt of claim 5, wherein U is —CNH₂, —CNRH, or —CNR₂.

7. The compound or salt of claim 1, wherein X and Y are independently selected from CR¹, N, NH, NR, O, S and P.

8. The compound or salt of claim 7, wherein X and Y are independently selected from N, NH, and NR.

9. The compound or salt of claim 1, wherein one or both of X and Y are substituted with a substituent selected from aryl, hydroxyl, thio, thioether, halide, ester, amide, carbonyl, thiocarbonyl, carboxyl, carboxylate and amino.

10. The compound or salt of claim 1, wherein Z is N—R.

11. The compound or salt of claim 1, wherein R¹, R², and R³ are independently selected from H, NH₂, NHR, OH and —OR.

12. The compound or salt of claim 1, comprising an unsaturated bond in the structure of formula 1.

13. The compound or salt of claim 1, wherein a substituted alkyl within the definition of R is substituted with one or more substituents selected from aryl, hydroxyl, thin, thioether, ether, amino, halide, ester, amide, carbonyl, thiocarbonyl, carboxyl and carboxylate.

14. The compound or salt of claim 13, wherein a substituted alkyl with the definition of R is substituted with O(CH₂)ₚH or OC(O)(CH₂)pH, wherein p is an integer from 1 to 10.

15. The compound or salt of claim 13, wherein a substituted alkyl within the definition of R is substituted with —NR⁵, wherein R⁵ is selected from hydrogen; substituted or unsubstituted aryl; and substituted or unsubstituted linear or branched alkyl.

16. The compound or salt of claim 15, wherein substituents of R⁵ are selected from ether, amino, hydroxyl, ester, thioether, amide, carbonyl, thiocarbanyl, carboxyl, carboxylate, sulphanate, nitro and halide.

17. The compound or salt or claim 1, wherein a substituted aryl within the definition of R is substituted with alkyl, aryl, acyl, ether, amino, hydroxy, ester, thioether, amide, nitro, cyano, carbonyl, carboxyl, carboxylate and halide.

18. The compound or salt of claim 1, wherein the compound 25 has the structure:

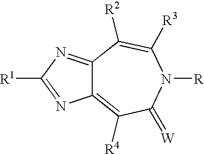

19. The compound or salt of claim 1, wherein R⁴ is a branched alkyl group or a substituted or unsubstituted aryl group.

20. The compound or salt of claim 19, wherein the compound has the 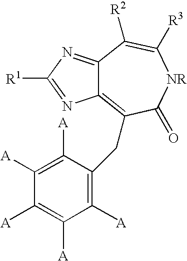
structure:
where A groups are independently selected from H and alkyl, aryl, acyl, ether, halide, thioether, hydroxyl, amino, nitro, cyano, and carboxylate substituents.

21. The compound or salt of claim 20, wherein one or more A substituents is —OR, —OH, or halide.

22. The compound or salt of claim 21, wherein the para-A substituent is —OR or OH and meta-A substituents are halide.

23. The compound or salt of claim 21, wherein —OR in an A substituent is —OCH₃.

24. The compound or salt of claim 21, wherein halide in an A substituent is Br.

25. The compound or salt of claim 18, wherein R¹ is an amine group.

26. The compound or salt of claim 1, having the structure of ceratamide A or a tautomer thereof.

27. The compound or salt of claim 1, having the structure of ceratamide B or a tautomer thereof.

28. Use of an antimitotic compound according to claim 1, for arresting cellular mitosis.

29. Use of an antimitotic compound according to claim 1, for preparation of an antimitotic agent or medicament.

30. A composition comprising an effective amount of an antimitotic compound according to claim 1, and a pharmaceutically acceptable carrier.

31. A method of arresting mitosis in a population of cells comprising contacting a population of cells with an effective amount of an antimitotic compound according to claim 1, whereby dividing cells in the population are arrested in mitosis.

32. The method of claim 31, wherein the contact is in vitro.

33. The method of claim 31, wherein the contacting is in vivo.
